(19) United States
(12) Patent Application Publication
(10) Pub. No.: US 2008/0262381 A1
(43) Pub. Date: Oct. 23, 2008

(54) Infant SID Monitor Based On Accelerometer

(75) Inventor: Paul T. Kolen, Encinitas, CA (US)

Correspondence Address: FISH & RICHARDSON, PC, P.O. BOX 1022, MINNEAPOLIS, MN, 55440-1022, US

(73) Assignee: MAGNETO INERTIAL SENSING TECHNOLOGY, INC., Carlsbad, CA (US)

(21) Appl. No.: 12/102,825

(22) Filed: Apr. 14, 2008

Related U.S. Application Data
(60) Provisional application No. 60/911,450, filed on Apr. 12, 2007.

(51) Int. Cl. A61B 5/11; A61B 5/01

(52) U.S. Cl. 600/549; 600/595

(57) ABSTRACT

Techniques, devices and systems that monitor the orientation and breathing of an infant and wirelessly communicate the orientation/breathing data to a caregiver through a wireless interface to request intervention if an unsafe situation is detected.

This application claims the benefit of U.S. Provisional Application No. 60/911,450 entitled “Infant SID Monitor Based On Accelerometer” and filed on Apr. 12, 2007, which is incorporated by reference as part of the specification of this application.

### BACKGROUND

This application relates to sensors, including sensors for monitoring of infants to detect and prevent Sudden Infant Death (SID) Syndrome.

SID is a spontaneous and unpredictable cessation of breathing by the infant resulting in death due to oxygen deprivation to the brain and other body organs. The scientific literature has shown that nearly 80% of the cases of SID are associated with the infant found in the face-down position, on the stomach, while sleeping. One reason for this association is not well understood but could be due to the lack of proper development associated with the breathing muscles.

### SUMMARY

This application describes techniques, devices and systems that monitor the orientation and breathing of an infant and wirelessly communicate the orientation/breathing data to a caregiver through a wireless interface to request intervention if an unsafe situation is detected. In one aspect, an infant monitor system include a infant sensor module; and a control module in wireless communication with the infant sensor module. The infant sensor module includes a base attachable to an infant; three accelerometers on the base to measure accelerations along three different directions, respectively; a signal processor on the base to receive outputs from the three accelerometers and to produce a sensor signal based on the outputs; and a sensor RF transceiver on the base in communication with the signal processor to wirelessly transmit the sensor signal to the control module. The control module includes a controller RF transceiver in wireless communication with the sensor RF transceiver; a controller processor operable to process the sensor signal to measure an orientation and a motion of the infant sensor module and to generate an alert when the measured orientation matches a pre-determined orientation for triggering the alert or when the measured motion matches a pre-determined motion profile for triggering the alert; and a communication interface to communicate the alert from the controller processor to a destination outside the control module.

In another aspect, a method for monitoring an infant includes attaching a sensor module to the infant to monitor an orientation and a motion of the infant sensor module by using three accelerometers in the sensor module to measure accelerations along three different directions, respectively; processing the outputs from the three accelerometers to produce a sensor signal; wirelessly transmitting the sensor signal to a control module; operating the control module to process the sensor signal to measure to generate an alert when the measured orientation matches a pre-determined orientation for triggering the alert or when the measured motion matches a pre-determined motion profile for triggering the alert; and sending the alert to a destination outside the control module.

Details of these and other aspects, implementations and examples are described in the drawings, the description and the claims.

### BRIEF DESCRIPTION OF DRAWINGS

FIG. 1A is an example of the infant mounted Sensor Module (SM).

FIG. 1B is an example of the local, short range RF control and signal processing module (CSPM).

FIG. 1C is an example of a short range RF monitoring module (MM) in lieu of long range alternatives shown in FIG. 1B.

FIG. 2 is an example of the analysis flowchart incorporated into the signal processing module described in 1B.

### DETAILED DESCRIPTION

A child on its stomach would require the infant to raise the weight of the upper body to inflate the chest cavity and lungs instead of merely working against the elasticity of the diaphragm and related body mass. This added load coupled with underdeveloped breathing related muscularity could inhibit the breathing action to the point where asphyxiation could result. Additionally, in the face down position, airflow obstacles like blankets and pillows near and around the infants face and mouth could cause the exhale and carbon dioxide level to increase exasperating the situation further.

A monitor capable of sensing the orientation of the infant while sleeping can be placed on the infant to generate an alert to a caregiver when the child positioned itself in the face-down position. This would inform the caregiver that the infant needs to be repositioned into a face-up position as soon as possible. The monitor can be configured to monitor the actual breathing of the infant to alert the caregiver if the infant stopped breathing for a pre-determined period of time regardless of the infant sleeping position.

This application describes, among others, techniques, devices and systems that monitor the orientation and breathing of an infant and wirelessly communicate the orientation/breathing data to a caregiver through a wireless interface to request intervention if an unsafe situation is detected. The monitoring system can include, in one implementation, a battery powered sensor module capable of monitoring both the static orientation of the sleeping child as well as monitoring the breathing. The infant mounted sensor module can incorporate a microprocessor, short-range RF transceiver link, and a battery power source to allow data acquisition, formatting and transmission.

The transmitted data from the infant mounted sensor module would be received by an associated analysis unit capable of the required digital signal processing to both determine the infant orientation as well as to detect the presence of normal breathing activities. If the analysis unit determines that intervention by a care giver is require, an alert is generated and transmitted to the caregiver through a variety of communication paths which are described in greater detail in the attached drawings and detailed description in the following text.

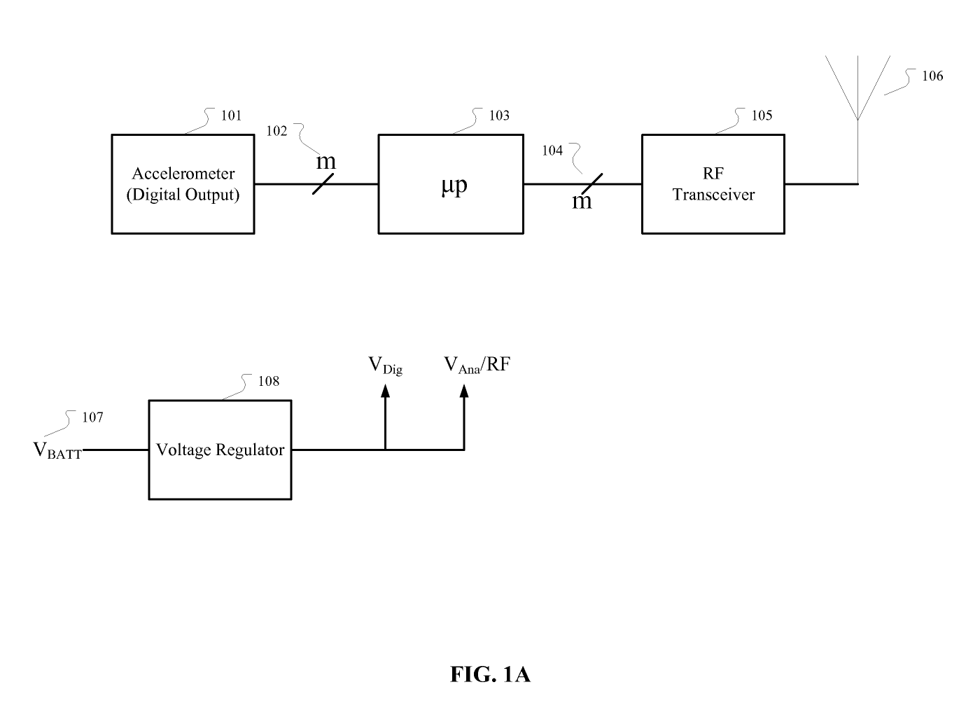

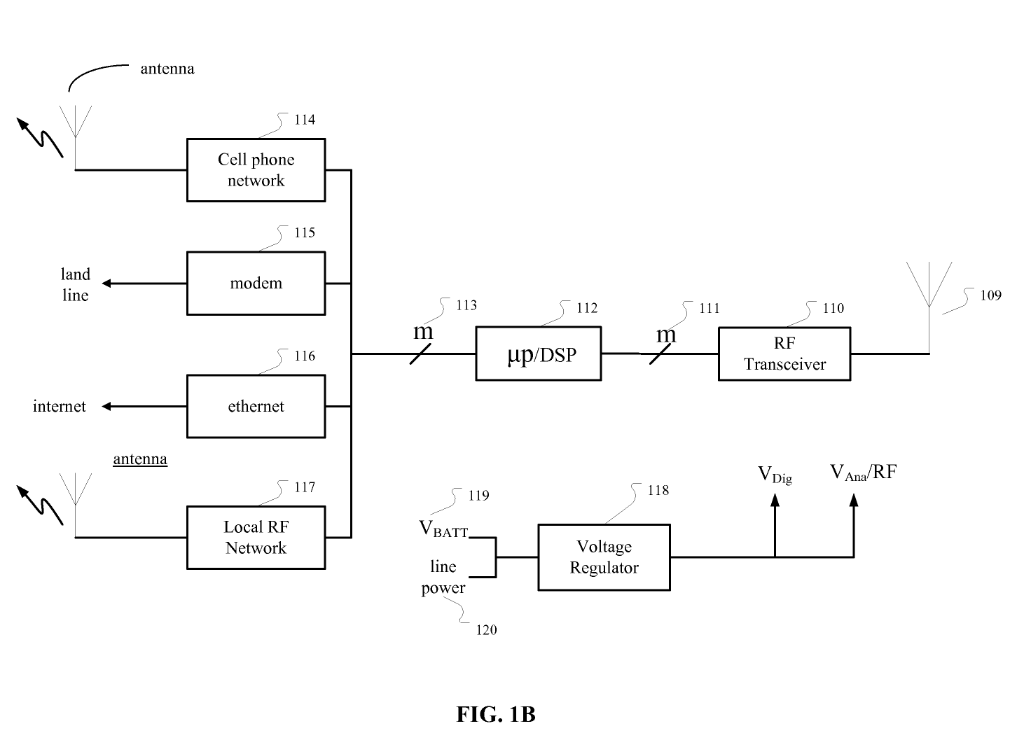

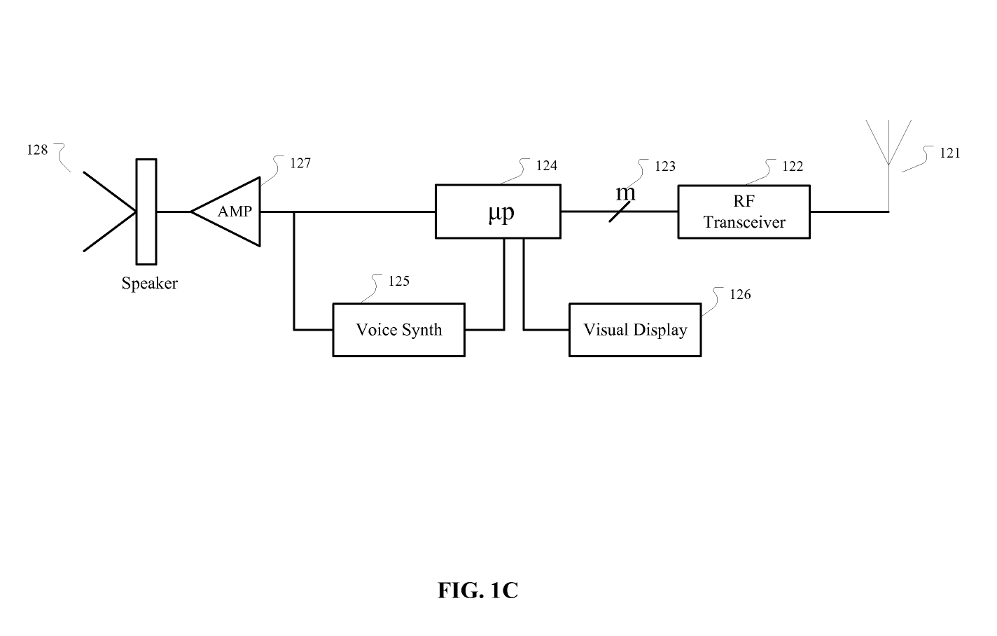

The system shown in FIGS. 1A-1C is an example of a sensing system that is configured to monitor conditions associated with SID Syndrome. Each sensor module for attaching to a child includes three accelerometers for measurements along three different directions (e.g., three orthogonal directions X, Y and Z) can be made in a compact package based on the Micro-Electro-Mechanical Systems (MEMS) technology using micro fabrication processes.

FIG. 1A shows an example sensor module (SM) for mounting on the stomach or chest area of an infant for monitoring the orientation and breathing activities. A processor 103 can be programmed to acquire data from an accelerometer 101 via a serial digital interface 102 between the accelerometer 101 and the processor 103. The processor 103 is programmed to remain in low power sleep state until a predetermined time has elapsed. Upon exiting the sleep state, the processor 103 can power up the accelerometer 101 and an RF transceiver 105 of the sensor module. Once powered, the processor 103 collects sensor data from the accelerometer 101 for a predetermined data sample period, i.e. 10 seconds of sampled data for every 60 second period resulting in a duty cycle of 17%. In one implementation, no signal processing of the accelerometer data collected from the accelerometer 101 is performed on the SM except to format the data for transmission to a control and signal processing module (CSPM) via the RF transceiver 105 and an integrated antenna 106. This operation can conserve battery energy and extend the operating time of the battery. The SM is powered via an integrated rechargeable primary battery 107 thru a linear voltage regulator 108. The formatted data is transferred to the RF transceiver 105 via a serial digital interface 104 for transmission to the CSPM.

FIG. 1B shows an example of a control and signal processing module (CSPM). The CSPM receives the current accelerometer data transmission from the SM via an integrated antenna 109, an RF transceiver 110, and a serial digital interface 111. Once received, the data is processed by the appropriate digital signal processing algorithms, to be described later, in a Processor/DSP engine 112. The digital processing of the accelerometer data can extract the infant orientation and detect the presence of breathing or the lack thereof. If the infant is not in a face-down orientation and breathing is detected, the CSPM can discard or save the data and wait for the next data transmission from the SM during the next sample period. However, if the CSPM determines that the infant is in a face-down position and/or no breathing is detected, an alert is broadcasted to the caregiver by any chosen combinations of available communication modes supported by the CSPM. Examples of the potential communication modes could include, but are not limited to, a cell phone via a cell phone network 114 using either text or speech, a modem 115 for transmission via land line, an Ethernet connection 116 for transmission via internet, or to a local, short range RF interface 117 to a dedicated monitor module (MM) shown in FIG. 1C. An example of this MM application would be to locate the MM in the parents bedroom while the CSPM is near the infant and SM. The CSPM is powered via a linear voltage regulator 118 with power provided by either a rechargeable/primary battery 119 or a power line 120. The CSPM can be configured to provide continuous status information about the infant, i.e., current infant orientation and breathing rate if desired.

FIG. 1C shows an example monitor module (MM). In the figure, a processor 124 communicates with the CSPM through the short range RF interface 117 from FIG. 1B via an integrated antenna 121, an RF transceiver 122, and a serial digital interface 123. If desired, the status information about the infant can be displayed on a visual display 126, i.e. an LCD screen. If the status information indicates a face-down condition or breathing problem, an audible alert can be generated by the processor 124 in the form of a tone or via a voice synthesizer 125. The tone and/or voice is amplified and transmitted by an integrated amplifier 127 and a loudspeaker 128.

In addition to using a “generic” model for normal and abnormal breathing, i.e. rapid breathing associated with crying, the processor 112 of the CSPM can be first operated in a learning mode to “learn” the normal breathing profile of the infant. While in the learn mode, the processor 112 establishes normal breathing parameters while the infant is known to be in a normal breathing state, i.e. non-crying. These stored parameters are then used to compare against the current breathing pattern to alert the local caregiver to the fact that the infant possibly needs attention. This learning capability can further be extended to monitor the actual body movement of the infant monitored by the accelerometer 101 in the SM to determine if the unusual motion requires attention by the caregiver.

In operation, the sensor data from the SM in FIG. 1A is received by the CSPM shown in FIG. 1B via the integrated antenna 109 and the RF transceiver 110. This data is then transferred to the processor/DSP 112 via the serial digital interface 111.

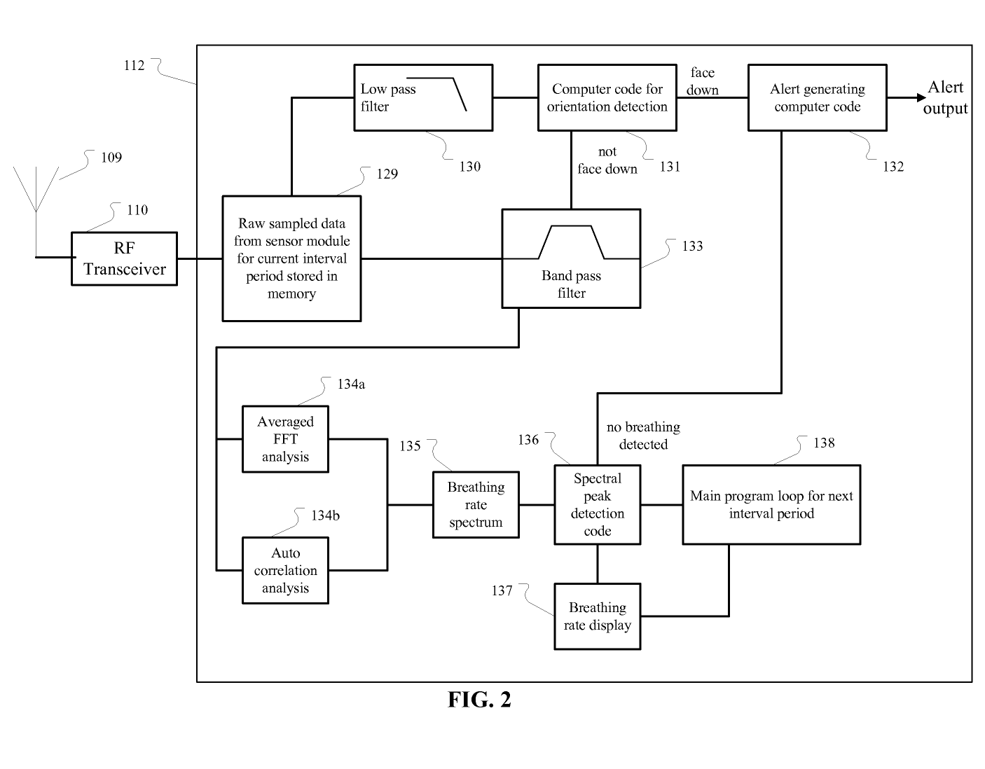

FIG. 2 shows an analysis flowchart of software used by the processor/DSP 112 to detect the infant position and breathing status from the raw accelerometer data. Once the data packet has been received by the processor/DSP 112, the data is parsed into separate arrays for the X, Y, and Z axis accelerometers and stored in a memory 129. Once stored, the accelerometer data is used to determine the static orientation of the infant. Each of the three accelerometer arrays are digitally filtered by a low pass filter function 130 to remove all high frequency content above approximately 2 Hz. The filtered data is then passed on to a routine 131 used to determine the physical orientation of the infant from the filtered data. If it is determined that the infant is in a face-down orientation, control is passed to a routine 132 used to generate an alert to all the communication modes that have been enabled in the system. If the routine 131 determines that the infant is in a safe orientation, a breathing detection algorithm is engaged.

The same data stored in the arrays used in the orientation algorithm can be used in the breathing detection algorithm. The data is digitally filtered by a band pass filter 133 to remove the low frequency and high frequency components of the data spectrum outside the expected range of breathing frequencies. The expected breathing rates have been experimentally determined to be approximately 1 breath every 4 second (0.25 Hz) on the low side to a maximum breathing rate of approximately 4 breaths per second (4 Hz). The filtered data is then passed onto a routine 134a to perform a fast Fourier transform (FFT). A technique of FFT averaging can be employed to increase the signal to noise ration (S/N) of the individual FFT outputs to improve the system sensitivity to the infant breathing. This increased sensitivity allows better detection of weak breathing and reduce the number of false alerts. This technique is very useful in increasing the (S/N) of the resulting averaged FFT but at the expense of the spectral resolution of the actual breathing rate. This is the ideal algorithm if a breathing/not breathing detection is desired. Alternatively, if the true breathing rate of the infant is desired, i.e. to determine if the breathing rate is 30 or 32 breaths per minute, an auto-correlation or auto-regressive algorithm 134b can be employed at the expense of requiring additional processing power in the processor/DSP 112. In either case, a breathing rate spectrum 135 can be produced. This spectrum is analyzed by a spectral peak detection algorithm 136 to either detect the presence/no presence of breathing or determine the actual breathing rate. If no breathing is detected, the control is passed to the routine 132 used to generate an alert to all the communication modes that have been enabled in the system. If breathing is detected, and the breathing rate information is desired, a breathing rate display algorithm 137 is updated prior to returning to a main program loop 138 for the next data sample. Alternatively, if no breathing rate information is required, the code returns directly to the main program loop 138.

In another implementation, the root mean square (RMS_ value of the accelerometer output signal can be used as a reliable indicator of the child's activity level for breathing detection as an alternative to or a supplemental to the above FFT approach. Monitoring the RMS value of the Z-axis accelerometer, for example, can be used. If the RMS level drops below a pre-determined threshold level determined by the CSPM when in the learn mode, an audio alert can be generated to alert to the possibility of a lack of breathing or an abnormally low activity level to be checked. Additionally, if the RMS level is higher then the threshold level by a certain amount, the RMS value indicates that the child is active or may be crying. Another alert, e.g., an audio alert signal, can also be generated. An audio alert is also issued if the child is determined to be sitting up or standing.

In some implementations, a skin temperature sensor may be attached to the child to monitor the skin temperature while sleeping. The normal high and low skin temperatures are stored in the system when in the learn mode. If the temperature goes above the high threshold value, an audio alert is generated to the possibility of an elevated temperature or fever condition. If the temperature is below the low threshold, an alert can be generated to indicate the sensor has been removed or it is not in good skin contact or other condition that may case the low temperature at the sensor.

In above examples, the loss of the sensor signal may be an indicator the sensor failure or that the child has been removed from the room and is out of RF range. A corresponding alert signal can be generated so that the operator can be dispatched to check on the status of the child and the sensor.

While this specification contains many specifics, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Only a few implementations are described. Other implementations, variations and enhancements may be made based on what is disclosed in this application.

What is claimed is:

1. An infant monitor system, comprising:
a infant sensor module; and
a control module in wireless communication with the infant sensor module,
wherein the infant sensor module comprises:
a base attachable to an infant;
three accelerometers on the base to measure accelerations along three different directions, respectively;
a signal processor on the base to receive outputs from the three accelerometers and to produce a sensor signal based on the outputs; and
a sensor RF transceiver on the base in communication with the signal processor to wirelessly transmit the sensor signal to the control module, and
wherein the control module comprises:
a controller RF transceiver in wireless communication with the sensor RF transceiver;
a controller processor operable to process the sensor signal to measure an orientation and a motion of the infant sensor module and to generate an alert when the measured orientation matches a pre-determined orientation for triggering the alert or when the measured motion matches a pre-determined motion profile for triggering the alert; and
a communication interface to communicate the alert from the controller processor to a destination outside the control module.

2. The system as in claim 1, wherein the three accelerometers in the infant sensor module are MEMS devices.

3. The system as in claim 1, wherein the communication interface in the controller module includes at least one of
a radio transceiver interface with a cell phone network,
a modem connected to a land line,
an Ethernet card with a computer network such as the Internet, and
an RF transceiver module to wirelessly communicate with a local RF network.

4. The system as in claim 1, wherein the controller processor performs signal filtering on digital data of the sensor signal and fast Fourier transform on the filtered digital data in generating the alert.

5. The system as in claim 1, wherein the controller processor computes a root mean square value of the sensor signal and compares the root mean square value of the sensor signal to a pre-determined threshold value in generating the alert.

6. The system as in claim 1, comprising a temperature sensor in contact with the infant to measure a skin temperature, wherein the controller processor is connected to receive the temperature measurement from the temperature sensor and to produce a temperature alert signal when the temperature is below a low temperature threshold or above a high temperature threshold.

7. A method for monitoring an infant, comprising:
attaching a sensor module to the infant to monitor an orientation and a motion of the infant sensor module by using three accelerometers in the sensor module to measure accelerations along three different directions, respectively;
processing the outputs from the three accelerometers to produce a sensor signal;
wirelessly transmitting the sensor signal to a control module;
operating the control module to process the sensor signal to measure to generate an alert when the measured orientation matches a pre-determined orientation for triggering the alert or when the measured motion matches a pre-determined motion profile for triggering the alert; and
sending the alert to a destination outside the control module.

8. The method as in claim 7, comprising:
attaching a temperature sensor in contact with the infant to measure a skin temperature; and
producing a temperature alert signal when the temperature is below a low temperature threshold or above a high temperature threshold.
